(19) United States
(12) Patent Application Publication
(10) Pub. No.: US 2020/0115671 A1
(43) Pub. Date: Apr. 16, 2020

(54) NANONEEDLE AND RELATED APPARATUS AND METHODS

(71) Applicant: NEEM SCIENTIFIC, INC., San Diego, CA (US)

(72) Inventor: DELI WANG, San Diego, CA (US)

(73) Assignee: NEEM SCIENTIFIC, INC.

(21) Appl. No.: 16/715,206

(22) Filed: Dec. 16, 2019

Related U.S. Application Data
(63) Continuation of application No. PCT/US2018/037825, filed on Jun. 15, 2018.
(60) Provisional application No. 62/521,276, filed on Jun. 16, 2017.

(51) Int. Cl. C12M 1/00; A61M 37/00; C12M 1/42; C12M 3/06

(52) U.S. Cl. CPC C12M 47/04; A61M 37/0015; C12M 35/00; B82Y 5/00; A61M 2037/003; A61M 2205/0244; C12M 23/16

(57) ABSTRACT

Disclosed herein are apparatus and methods to perform in-vitro probing of cell interior using a nanoneedle. Some aspects of the present application relate to an apparatus with a vertical nanoneedle disposed in a flow channel, wherein the flow channel is shaped to facilitate immobilization of a cell recirculating in a fluid in the flow channel with the nanoneedle and penetration of the cell membrane with the nanoneedle. Aspects of the present application also provide an integration between the flow channel and a cell sorter to form a medical system that selectively and continuously communicates intracellularly with screened cells of interests.

### CROSS-REFERENCE TO RELATED APPLICATIONS

This application is a continuation of international patent application PCT/US2018/037825 filed Jun. 16, 2018, further claims priority under 35 U.S.C. § 119(e) to U.S. Provisional Application No. 62/521,276, filed Jun. 16, 2017, which is incorporated herein by reference in its entirety for all purposes.

### BACKGROUND

In-vitro probing of cell interior is typically performed by immobilizing or culturing a cell on a substrate, followed by inserting a probe such as a patch clamp into the interior of the cell.

### SUMMARY

According to some embodiments, an apparatus is provided. The apparatus comprises a first flow channel configured to accommodate a first fluid containing a cell circulating along a first flow direction; a nanoneedle disposed in the first flow channel and configured to penetrate the cell. The first flow channel comprises a constriction adjacent the nanoneedle.

According to some embodiments, a method of manufacturing a nanopump apparatus is provided. The method comprises forming a nanoscale wire; forming a side wall material surrounding the nanoscale wire; disposing the nanoscale wire inside a first flow channel; and subsequent to disposing the nanoscale wire inside the first flow channel, selectively removing the nanowire to form a nanoneedle from the side wall material.

According to some embodiments, a method of operating an apparatus is provided. The apparatus includes a first flow channel, a second flow channel, a nanoneedle that comprises a first opening disposed in the first flow channel and a second opening disposed in the second flow channel. The first flow channel comprises a constriction adjacent the nanoneedle. The method comprises circulating a first fluid containing a cell along a first flow direction in the first flow channel; penetrating the cell with the nanoneedle; accommodating a second fluid containing a reagent in the second flow channel; and delivering the reagent from the second fluid into the cell via the nanoneedle.

According to some embodiments, a medical system is provided. The medical system comprises a nanopump comprising a first flow channel, a second flow channel having a fluid containing a reagent, a nanoneedle that comprises a first opening disposed in the first flow channel and a second opening disposed in the second flow channel. The first flow channel is configured to receive a first sample containing a cell from a user. The nanoneedle is configured to penetrate and deliver the reagent inside the cell.

### BRIEF DESCRIPTION OF DRAWINGS

Various aspects and embodiments will be described with reference to the following figures. The accompanying drawings are not intended to be drawn to scale. In the drawings, each identical or nearly identical component that is illustrated in various figures is represented by a like numeral. For purposes of clarity, not every component may be labeled in every drawing.

FIG. 1A is a schematic diagram illustrating a cross-sectional view of an apparatus 100 having a nanoneedle, according to some embodiments;

FIG. 1B is a schematic diagram illustrating a cross-sectional view of apparatus 100 within the first portion 126 taken along the vertical plane A-A′ as shown in FIG. 1A;

FIG. 2A is a schematic diagram illustrating a top view of an apparatus 200, according to some aspects;

FIG. 2B is a schematic diagram illustrating a top view of the cover 206 of the first flow channel 220 of apparatus 200, according to some aspects;

FIG. 3A is a schematic diagram illustrating a top view of an apparatus 300A having multiple nanoneedles 302 arranged in a regular two-dimensional array in base 304;

FIG. 3B is a schematic diagram illustrating a top view of an apparatus 300B having multiple nanoneedles 302 arranged in a random two-dimensional array in base 304;

FIG. 3C is a schematic diagram illustrating a top view of an apparatus 300C having multiple nanoneedles 302a, 302b, 302c and 302d arranged in base 304;

FIG. 4A and FIG. 4B are schematic diagrams illustrating an exemplary fabrication sequence to form a bottom channel 430 in a substrate 410, according to some aspects;

FIGS. 5A-5E are schematic diagrams illustrating an exemplary fabrication sequence to form a nanoneedle 502 on a base 504 in an apparatus of the type shown in FIGS. 1A and 1B;

FIGS. 6A and 6B are schematic diagrams illustrating a top view of an exemplary fabrication sequence for a first flow channel, according to an aspect of the present application;

FIGS. 7A-7C are schematic diagrams illustrating an exemplary fabrication sequence to form a nanoneedle and attach the nanoneedle to the first and second flow channels, according to some aspects;

FIGS. 8A and 8B are schematic diagrams illustrating a cross-section view of a fabrication sequence to form a nanoneedle array and attach the nanoneedle array to the first and second flow channels, according to some embodiments of the present application;

FIGS. 9A is a schematic diagrams illustrating a cross-sectional view of an apparatus 900, according to an alternative embodiment;

FIG. 9B is a schematic diagrams illustrating a top view of apparatus 900;

FIGS. 10A-10J are schematic diagrams illustrating an exemplary fabrication sequence to form apparatus 900, according to some aspects;

FIG. 11 is a schematic diagram showing a cross-sectional view of an apparatus 1100 with a nanopump 1102 and a piezoelectric membrane driver 1112, according to an aspect of the present application;

FIG. 12A is a schematic diagram showing a cross-sectional view of a nanopump 1202, according to an aspect of the present application;

FIG. 12B is a schematic diagram showing a cross-sectional view of nanopump 1202 according to the embodiment shown in FIG. 12A under a bias voltage;

FIG. 13 is a schematic diagram showing an exemplary medical system 1300 that includes a nanopump apparatus 1340, according to an aspect of the present application;

FIG. 14 is a schematic diagram showing an exemplary medical system 1400, according to another aspect of the present application;

FIG. 15 is a schematic diagram showing an exemplary apparatus 1500, according to an aspect of the present application.

### DETAILED DESCRIPTION

Intracellular probing to inject into, extract from, or otherwise communicate electrochemically with the cell interior may provide a broad range of applications in in-vitro diagnosis, therapeutics as well as brain research. The inventor has recognized and appreciated that one approach to improve throughput is to provide a continuous circulation of cells within a flow channel comprising one or more nanoneedles to allow continuous operation of intracellular probing. Aspects of the present application provide an integration between the flow channel and a cell sorter to form a medical system that selectively and continuously communicates intracellularly with screened cells of interests from a patient's blood sample.

Some aspects of the present application relate to an apparatus with a vertical nanoneedle disposed in a flow channel, wherein the flow channel is shaped to facilitate immobilization of a cell recirculating in a fluid in the flow channel with the nanoneedle and penetration of the cell membrane with the nanoneedle. The inventor has recognized and appreciated that a region of the flow channel where the nanoneedle is disposed may have a reduced dimension in the width and/or height direction that is substantially the same or smaller than a size of the cell. In this fashion, the flow channel is configured such that the cell has a tight fit or is slightly squeezed when flowing (or passing) over the nanoneedle, which is in the flow path of the cell, to facilitate insertion of a tip of the nanoneedle into a cell. According to some aspects, the region of the flow channel is further sized to have a reduced dimension in the width and/or height direction that is substantially the same or smaller than a size of the cell nucleus such that the nanoneedle is in the flow path of a cell nucleus, to facilitate insertion of the nanoneedle inside the cell nucleus. According to yet another aspect, the flow channel may be transparent to allow imaging of the cells inside the flow channel as they flow across the nanoneedle. Imaging feedback may be used in conjunction with the flow control to facilitate cell immobilization and penetration.

Some aspects of the present application relate to a method to fabricate a nanoneedle. The nanoneedle may have a hollow, tube-shape structure formed by depositing a material of the nanoneedle side wall over a sacrificial template. The sacrificial template may be a vertical nanowire with a cross-section dimension defining the interior cross-section of the nanoneedle after the sacrificial template is removed. The nanoneedle side wall material may be selected and may be further functionalized to facilitate cell immobilization and cell membrane penetration. The cross-section shape and dimension of the tip of the nanoneedle are configured to allow penetration of the cell membrane and/or into the cell nucleus with the cell remaining substantially viable. In this fashion, a whole-life study of a cell interior may be performed.

According to some aspects, the nanoneedle is vertically oriented and disposed in a horizontal first flow channel. A tip opening of the nanoneedle is in fluidic communication with the liquid in the first flow channel, or when a cell is immobilized on the tip, with the intracellular fluid of the cell in the flow channel. In some embodiments, a second flow channel may be provided underneath the nanoneedle and connected to the interior of the nanoneedle. In one embodiment, the nanoneedle may extract analytes from the cell interior to be delivered to the second flow channel for diagnostic analysis. In another embodiment, the nanoneedle may deliver material from the second flow channel to be injected into the cell interior or inside the nucleus, for example to perform therapeutic drug delivery.

The inventor has recognized and appreciated various methods may be provided to allow controlled pumping of fluids in and out of a cell via the nanoneedle, thus using the nanoneedle as a nanopump. In one aspect, electrode materials may be provided along the nanoneedle sidewalls and at the base of the nanoneedle to control liquid flow using for example electrowetting effect. In another aspect, a piezoelectric driving module may be provided in one of the flow channels to drive the nanopump liquid flow. According to some aspects, controlled pumping via the nanoneedles into or out of a cell interior may allow synchronized fluidic communication in accordance with a predetermined timing and dosage. According to yet another aspect, the controlled pumping may be used to facilitate selective attachment and detachment of cells on a nanoneedle.

According to some aspects, a plurality of nanoneedles may be provided in a region of a flow channel. Each one of the nanoneedles may be configured to be connected to the same or different liquids. In one example, a column of nanoneedles may be disposed along the flow direction inside a flow channel such that a cell may be attached to a first nanoneedle, subsequently detached from the first nanoneedle and then attached to a second nanoneedle, and so on in a repeating fashion for a plurality of nanoneedles. One or more, and in some cases each, of the nanoneedles may be configured to inject a select sequence of biochemical molecules of certain dosage into the same cell to perform for example therapeutic drug delivery. One or more, and in some cases each, of the nanoneedles may be configured to inject a select sequence of biochemical molecules into more than one cells that are sequentially or simultaneously attached to the nanoneedles.

According to some aspects, a medical system may be provided that comprise a cell sorter that extracts and separate cells from a human patient's blood stream or body fluid, a nanoneedle in a flow channel to communicate with the interior of the cells circulated from the cell sorter and to perform for example genetic editing or drug delivery to the cell or nucleus interior. In one example, the gene-edited cell may be cultured and infused back to the human patient by self-transplantation. According to some aspects, a self-transplantation system comprising the cell sorter and the nanoneedle and flow channel apparatus may form a cancer immunotherapy machine.

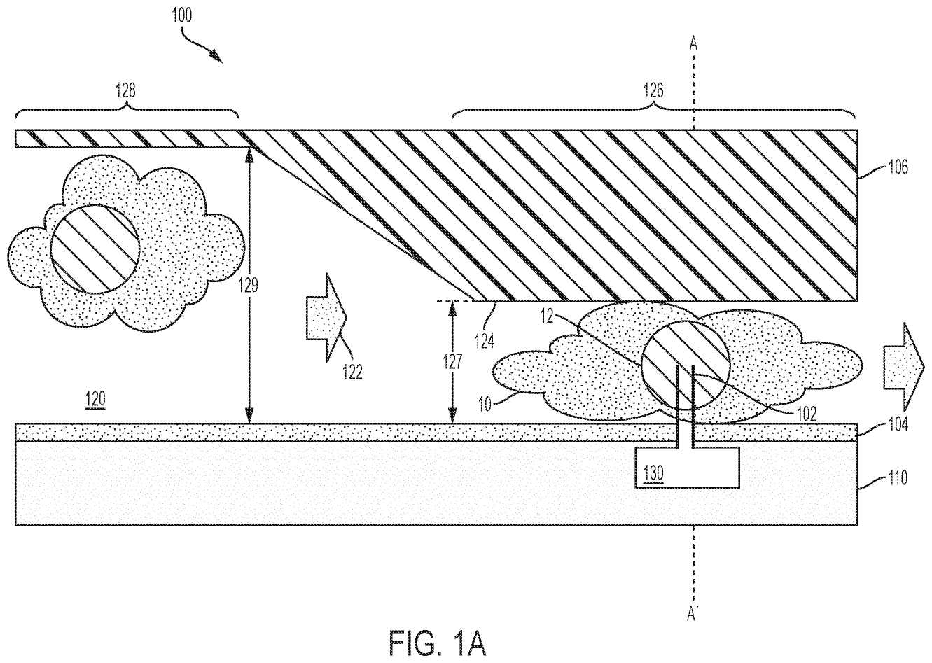

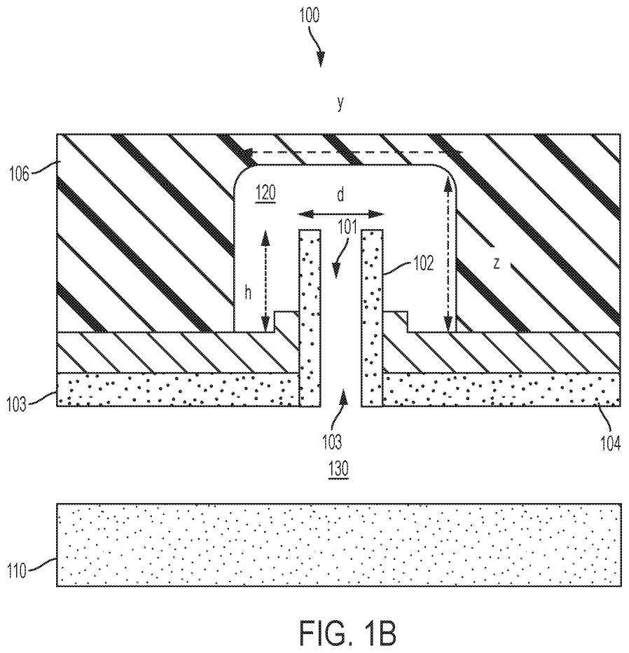

FIG. 1A is a schematic diagram illustrating a cross-sectional view of an apparatus 100, according to some embodiments. Apparatus 100 comprises a nanoneedle 102 vertically disposed in a first flow channel 120 and in fluidic communication inside a nucleus 12 of a cell 10, according to an aspect of the present application. The apparatus 100 further comprises a second flow channel 130 disposed in substrate 110 and in fluidic communication with the nanoneedle 102. In some embodiments, the first flow channel 120 may be referred to as a cell flow channel, the nanoneedle 102 is a nanopump and the second flow channel 130 may be referred to as a nanopump flow channel such that the nanopump 102 pumps fluid from the second flow channel 130 into the cell 10 or cell nucleus 12.

As shown in FIG. 1A, cell 10 is contained by a fluid (not shown) within the first flow channel 120, and travels along flow direction 122. In some embodiments, nanopump 102 may pump fluid from the nanopump flow channel 130 upward towards the first flow channel 120. First flow channel 120 comprises a constriction 124 as shown in FIG. 1A. First flow channel 120 has a first portion 126 within the constriction 124, and a second portion 128 outside the constriction. As shown in FIG. 1A, because constriction 124 protrudes inward of the flow channel, the first portion 126 has a smaller size 127 to allow liquid to flow through compared to a larger size 129 at the second portion 128 outside the constriction 124. The size 127 of the first portion 126 may be a distance orthogonal to the flow direction 122, such that the cell 10 is directed to flow adjacent to the nanoneedle 102 when the cell 10 enters the constriction 124, thus enhancing the probability for the nanoneedle 102 to interact with the cell 10. It is recognized that constriction 124 is preferably disposed adjacent nanoneedle 102 to direct flow of cell 10 such that cell penetration by nanoneedle 102 is facilitated. By adjacent, nanoneedle 102, constriction 124 may be above, next to, surrounding, or generally disposed within a distance of less than 100 μm, less than 50 μm, or less than 10 μm from the nanoneedle in some embodiments.

The sizes 127 and 129 may be a height of the first flow channel at the first portion 126 and the second portion 128 between a cover 106 and a base 104, respectively, as shown in FIG. 1A. Although it should be appreciated that the direction of the sizes 127/129 are not limited as the first flow channel 120 may be narrowed at the constriction 124 in any direction, such as along a lateral direction parallel to a surface of the base 104 that forms the bottom of the flow channel 120. It's also recognized that the constriction 124 may also take any appropriate shape along the flow direction to restrict the fluidic flow, and that it is not a requirement that the size 127 at the first portion 126 be uniform along the flow direction 122. In some embodiments, constriction 124 may be formed as part of the cover 106, although it should be appreciated that constriction 124 may be formed as a separate structure with a different material from cover 106.

In some embodiments, the constriction 124 is configured to facilitate insertion of nanoneedle 102 inside of cell 10, or inside a cell nucleus 12 of cell 10. Cell 10 may be an animal cell, a plant cell, a bacteria cell, or a fungi cell. Cell 10 may be a biological live cell, or a cell-like biological vesicle such as microbiota. Any appropriate size of the first size 127 at the first portion 126 of the first flow channel 120 may be provided to facilitate cell insertion. In some embodiments, the first size 127 may be between 0.25 and 5 times, between 0.5 and 2 times, or between 0.25 and 10 times the average diameter of cell 10. As used herein, average diameter of cell 10 may be an average of measured lateral extent of a single cell along multiple measurement axes in the event that the single cell may be irregularly shaped, or it may refer to an average of diameters measured for a group of cells of interest. Any suitable cell size measurement as known in the art may be used to determine the average diameter of cell 10. The first size 127 may be between 0.2 and 300 μm, between 0.25 and 200 μm, between 0.5 and 100 μm, between 1 and 100 μm, or between 10 and 100 μm.

In some embodiments, cell 10 is physically deformed within the first portion 126 in part due to the constriction 124 and/or penetration from nanoneedle 102, while the structure and function of cell 10 remains intact. In some embodiments, the first flow channel 120 may be a microfluidic channel and a fluid containing one or more cells 10 is continuously circulated within first flow channel 120 for the nanoneedle 102 to be able to penetrate multiple cells 10.

When nanoneedle 102 penetrates cell 10, a defined penetration depth within cell 10 may be selectively controlled by a variety of means, for example with a predetermined nanoneedle height relative the size of the cell. In some embodiments, cell 10 is contained in a first fluid within the first flow channel 120 and nanoneedle 102 may selectively deliver a second fluid containing one or more reagents (not shown) accommodated in second flow channel 130 inside the membrane or cell wall of cell 10 or inside cell nucleus 12, with selectively controlled quantity, flow rate, time duration. The injection may also include a substance representative of a needle ID. Contents in the second liquid can include but not limited to drug, small molecules, genome, growth factor, nucleic acids, protein, lipids, genome editing package, CRISPR formula, RNA, or a combination thereof. Intracellular injection by a nanoneedle in such a fashion may allow cellular level diagnostic or therapeutic applications related to cancer, HIV, or other diseases.

In some embodiments, in addition to delivery of extraneous substance inside of cell 10, nanoneedle 102 may also extract fluid from inside a penetrated cell. Contents of extracted intracellular fluid may pass through the nanoneedle 102 to the second flow channel 130 for further analysis of analytes contained in the intracellular fluid. In some embodiments, extraction of intracellular fluids may allow molecular level detection, live cell monitoring, drug discovery, and may assist research in genome editing, single cell research, cancer research, HIV, etc.

In some embodiments, injection/extraction via nanoneedle 102 may be driven by external pressure applied to fluid inside the nanoneedle. Driving of the external pressure may be by a piezoelectric device, a microelectromechanical system (MEMS) actuator or pump, or by electrowetting. In some embodiments, injection/extraction via nanoneedle 102 may be performed as a continuous operation.

Although FIG. 1A shows a single nanoneedle 102 within apparatus 100, it should be appreciated that multiple nanoneedles 102 may be provided within apparatus 100 to allow sequential injection, extraction, or the combination thereof. Multiple agent injection, time-lapse analysis, or real time bio-reaction and analysis may also be provided.

Apparatus 100 as shown in FIG. 1A may be integrated with a cell circulation device such as a cell sorter to form a medical system that allows continuous flow of cells through the first flow channel 120 for penetration by one or more nanoneedles 102 within apparatus 100 for therapeutic and diagnostic applications related to blood related diseases, cancer, HIV, etc. Integration with a cell line, a standalone high through-put device can be provided with the apparatus 100 for cell editing and modification, which can be associated with gene editing techniques such as CRISPR. A medical system comprising apparatus 100 may also be used for stem cell research, bio-reactor, drug discovery, and agriculture, etc.

In some embodiments, single nanopump/nanoneedle or an array of nanopump/nanoneedles may comprise either a rigid or flexible material, and can be used for brain research and disease treatment, including diagnosis and therapeutics, as well as microbiome research, diagnosis, and therapeutics.

FIG. 1B is a schematic diagram illustrating a cross-sectional view of apparatus 100 within the first portion 126 taken along the vertical plane A-A′ as shown in FIG. 1A. FIG. 1B shows that a width of the first flow channel 120 adjacent the nanoneedle is FIG. 1B shows that a width of the first flow channel 120 adjacent the nanoneedle is y, a height of the first flow channel 120 between cover 106 and a top surface of base 104 is z, a height of nanoneedle 102 between a first end 101 inside first flow channel 120 to a second end 103 inside second flow channel 130 is h, and a diameter of the nanoneedle is d. Nanoneedle 102 is a hollow structure comprising a side wall material, where both ends 101 and 103 are openings in fluidic communications with first flow channel 120 and second flow channel 130, respectively.

In some embodiments, height z may be the first size 127 configured to direct cell flow at constriction 124. Alternatively or in addition, width y may be the first size 127 configured to direct cell flow at constriction 124.

In some embodiments, nanoneedle height h may be adjustable based on the penetration depth needed to inject into/extract from just inside a cell membrane, or into a cell nucleus. The nanoneedle height h may be between 0.2 and 1000 μm, between 0.25 and 500 μm, between 0.5 and 200 μm or between 1 and 100 μm.

In some embodiments, nanoneedle diameter d may be an inner diameter or an outer diameter of nanoneedle 102, and may be selected based on the factors such as the quantity and flow rate to be injected, and the size and type of penetrated cell 10. The nanoneedle diameter d may be between 2 and 2000 nm, between 2 and 1500 nm, between 5 and 1500 nm or between 5 and 1000 nm.

Although FIG. 1B shows the second flow channel 130 underneath the nanoneedle extends substantially through the lateral extent of the base 104, such an arrangement is by way of example only and is not limiting. In some embodiments, the second flow channel 130 may be provided only under a portion of the base such that the rest of the base 104 outside the second flow channel 103 are supported by substrate 110 to prevent the nanoneedle 102 from collapsing into the second flow channel 130 below.

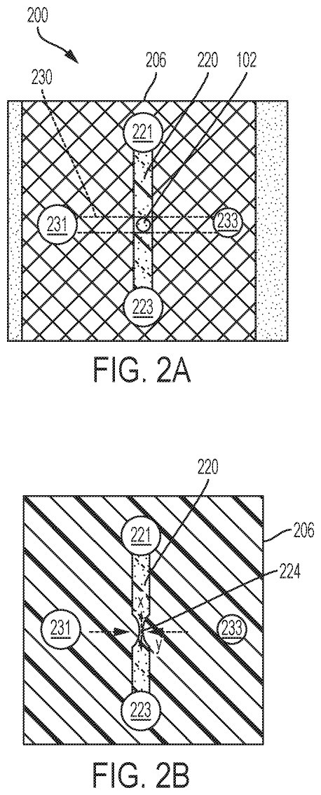

FIG. 2A is a schematic diagram illustrating a top view of an apparatus 200, according to some aspects. Apparatus 200 is similar to apparatus 100 in many aspects. Apparatus 200 comprises a nanoneedle 102 disposed vertically disposed within a first flow channel 220. Apparatus 200 further comprises a second flow channel 230 in fluidic communication with the nanoneedle 102. First flow channel 220 may be formed in a cover 206, and comprises ports 221 and 223 configured for circulation of a first fluid containing cells, from for example a media pump or a cell sorter. Second flow channel 230 comprises ports 231 and 233 configured for circulating a second fluid for injection to a cell via nanoneedle 102, or for delivering extracted intracellular fluid from the nanoneedle 102 for measurement and analysis. In some embodiments, ports 231 and 233 extend vertically through the cover 206 to provide fluidic access to the second flow channel 130.

FIG. 2B is a schematic diagram illustrating a top view of the cover 206 of the first flow channel 220 of apparatus 200, according to some aspects. Apparatus 200 has a constriction 224 within the first flow channel 220. In the first flow channel 220, liquid is configured to flow along a flow direction between ports 221 and 223. Constriction 224 may protrude inward to narrow a width y of the flow channel to direct cells to be penetrated by nanoneedle 102, and has a length X. The length X may be between 1 and 500 μm, between 1 and 200 μm, between 2 and 200 μm or between 5 and 200 μm.

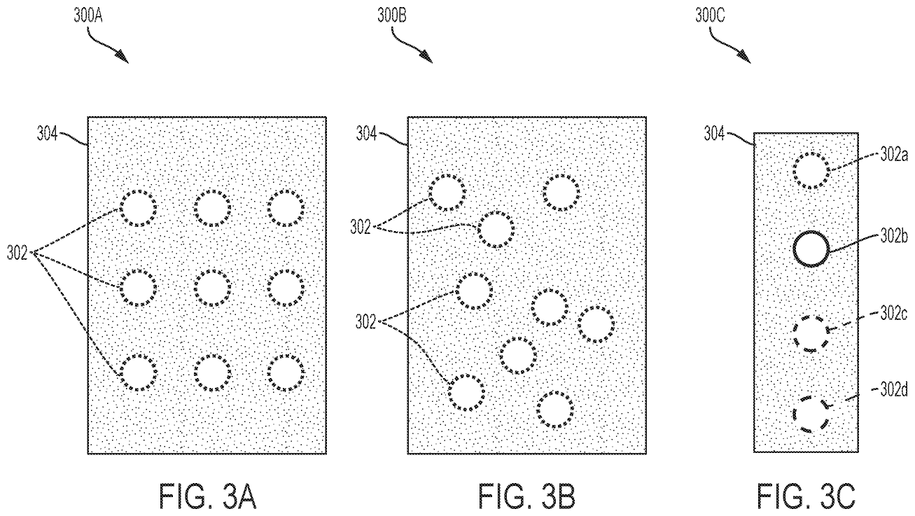

In some embodiments, multiple nanoneedles may be provided in an apparatus and arranged in a regular array. For example, FIG. 3A is a schematic diagram illustrating a top view of an apparatus 300A having multiple nanoneedles 302 arranged in a regular two-dimensional array in base 304. The array of multiple nanoneedles 302 may have the same height h extending into a first flow channel. In some embodiments, at least some of the array of nanoneedles 302 may have different heights h, and are configured to penetrate into different depths when a cell in a first flow channel is attached to the array of nanoneedles, or to penetrate into different depths of more than one cells.

In some other embodiments, multiple nanoneedles may be arranged in a randomly distributed array. For example, FIG. 3B is a schematic diagram illustrating a top view of an apparatus 300B having multiple nanoneedles 302 arranged in a random two-dimensional array in base 304.

FIG. 3C is a schematic diagram illustrating a top view of an apparatus 300C having multiple nanoneedles 302a, 302b, 302c and 302d arranged in in base 304. Although four nanoneedles are shown in FIG. 3C in the exemplary apparatus 300C, it should be appreciated that any number of multiple nanoneedles may be present in an apparatus according to aspects of the present application. In FIG. 3C, nanoneedles 302a, 302b, 302c, 302d may be identical, or may have different material composition, height, inner diameters and/or outer diameters. The nanoneedles also may be connected to more than one flow channels (not shown). For example, nanoneedle 302a may be connected to a first nanopump flow channel underneath base 304 and configured to inject a first type of fluid while penetrating a cell, while nanoneedle 302b may be connected to a second nanopump flow channel and configured to inject a second type of fluid, while penetrating the same cell. In this fashion, selective delivery inside the cell of different reagents contained in the first and second type of fluids at respective dosages may be provided. Alternatively or in addition, nanoneedle 302c may be connected to a third nanopump flow channel be configured to extract intracellular fluid from a cell penetrated by nanoneedle 302a, such that simultaneous delivery of reagents inside the cell by nanoneedle 302a, and extraction of intracellular fluid of the same cell by nanoneedle 302c may be provided. Simultaneous intracellular delivery and extraction may enable real-time monitoring of biochemical responses at a single-cellular level. It should be appreciated that any combination of nanoneedle arrays configured to inject into or extract from one or more cells may be provided on a same base within an apparatus.

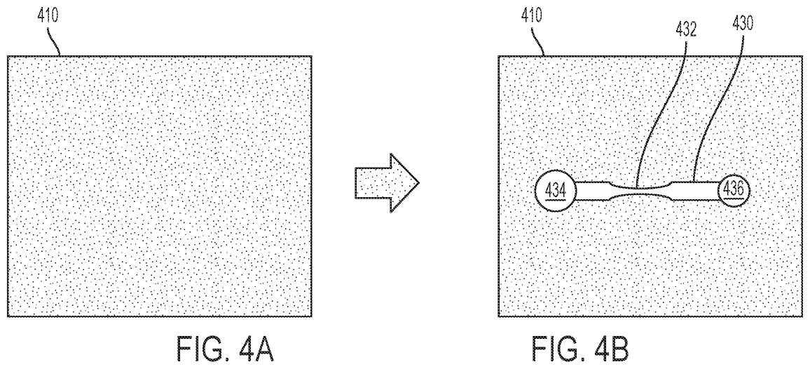

FIG. 4A and FIG. 4B are schematic diagrams illustrating an exemplary fabrication sequence to form a bottom channel 430 in a substrate 410, according to some aspects. The process may start with a substrate 410 as shown in FIG. 4A. Substrate 410 may be a single layer wafer, or may be a composite of multiple layers. In some embodiments, substrate 410 may comprise a semiconductor material such as but is not limited to Si or silicon oxide, such that standard microfabrication techniques can be used for subsequent fabrication steps.

In FIG. 4B, standard photolithography or electron beam lithography may be used to pattern and etch remove a portion of substrate 410 to define a second flow channel 430 with ports 434 and 436 configured to providing liquid access for circulation of fluid in the second flow channel 430. Second flow channel 430 may have any suitable depth and width for reagent or analyte flow. In some embodiments, second flow channel 430 may have a width of between 20 and 200 μm, between 50 and 200 μm, or between 50 and 100 μm. In some embodiments, second flow channel 430 may have a depth of between 20 and 200 μm, between 50 and 200 μm, or between 50 and 100 μm. A portion 432 of the second flow channel 430 is configured for a nanoneedle to be disposed thereabove, The second flow channel 430 may have a uniform width across the portion 432 with the rest of the second flow channel 430, or portion 432 may have a suitable shape and dimension different from the rest of the second flow channel 430 to facilitate reagent or analyte flow to or from the nanoneedle.

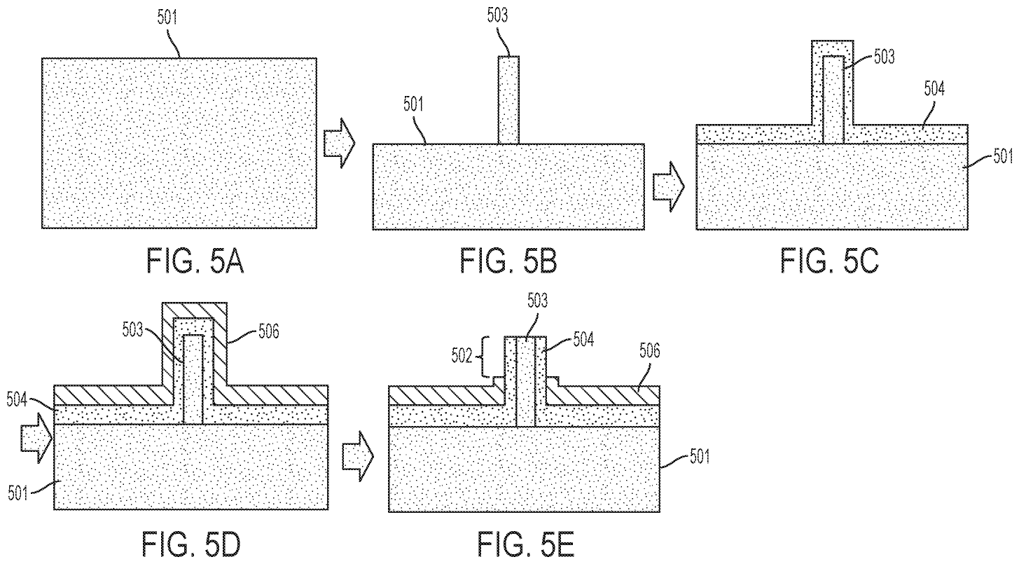

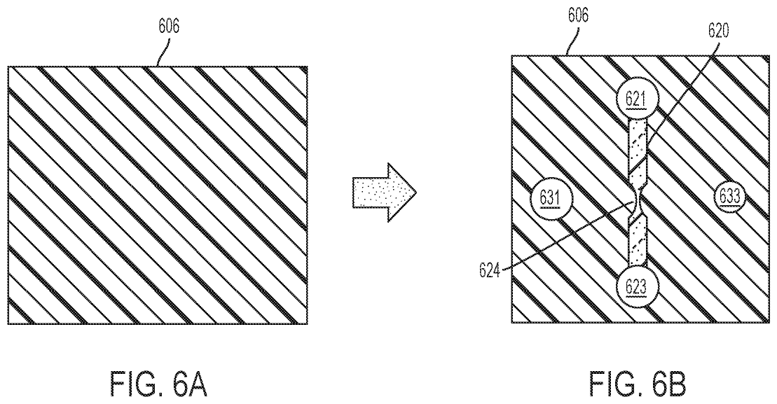

FIGS. 5A to 5E are schematic diagrams illustrating an exemplary fabrication sequence to form a nanoneedle 502 on a base 504 in an apparatus of the type shown in FIGS. 1A and 1B. The process may start with a wafer 501 as shown in FIG. 4A. Base 501 may be a single component wafer, or may be a composite of multiple layers. In some embodiments, wafer 501 may comprise a semiconductor material that is etchable by a suitable semiconductor etching technique. In a non-limiting example, base 501 is a Ge wafer.

In FIG. 5B, a nanowire 503 is formed vertically on a top surface of Ge wafer 501. Any suitable micro- or nano-fabrication technique may be used to form nanowire 503. For example, nanowire 503 may be formed by depositing a uniform layer of material and using a mask to anisotropically etch remove a portion of the materials outside the nanowire 503. Alternatively, nanowire 503 may be formed using a bottom-up growth technique as known in the art. For example, a vapor-liquid-solid growth or a vapor-solid growth process may be used. In one non-limiting example, nanowire 503 may be formed by anisotropically etching down the Ge wafer 501 with a etch mask that protects the cross-sectional area of nanowire 503 from etching. Etching may be done using a wet etch or a dry etch, and preferably an anisotropic reactive ion etch (ME). The formed nanowire 503 may have any suitable cross-sectional shape, with a diameter of between 20 and 300 nm, between 50 and 200 nm, or between 100 and 200 nm. The length of the nanowire 503 may be controlled by the etch depth from the original wafer 501, and may have a value of between 5 and 10 μm. The cross-sectional shape and diameter of the nanowire 503 determines the inner diameter and shape of the eventual nanoneedle after the nanowire 503 is sacrificially removed.

In FIG. 5C, a conformal layer of base material 504 is deposited and covers the top of wafer 501 as well as side wall and top of nanowire 503. Base material 504 may be a semiconductor material, and preferably a solid semiconductor material with sufficient mechanical rigidity to support the eventual nanoneedle formed on the base 504. In one non-limiting example, base material 504 comprises silicon oxide.

In FIG. 5D, layer 506 is deposited that covers the top surface of base 504 as well as the side wall and top of base material 504 over the nanowire 503. Layer 506 may be a material that provides protection to the layers below as well as compatibility to liquid in the first flow channel. In some embodiments, layer 506 may comprise a polymer such as polydimethylsiloxane (PDMS), or an epoxy resin such as SU-8.

In FIG. 5E, an etching is performed to selectively etch back a portion of layer 506 and base material 504 over the top of the tip of nanowire 503, such that the nanowire 503 is exposed on its top surface.

FIGS. 6A and 6B are schematic diagrams illustrating a top view of an exemplary fabrication sequence for a first flow channel 621, according to an aspect of the present application. The process may start with a cover material 606 as shown in FIG. 6A. Some aspects of the application provide a first flow channel 621 fabricated on a cover material that is a semiconductor substrate such as Si or SiO₂, or a flexible molded polymer substrate, although it should be appreciated any suitable substrate may be used. In one non-limiting example, cover material 606 comprises PDMS.

In FIG. 6B, a first flow channel 620 is formed at a bottom surface of cover 606. In some embodiments, the first flow channel 620 have a width of between 50 and 100 μm. The first flow channel 620 comprises a constriction 624 configured for a nanoneedle to be placed therein. Any suitable shape for constriction 624 may be used, with a size in accordance to the discussion related to apparatus 100 shown in FIGS. 1A and 1B. The transition from the rest of the first flow channel 620 to the constriction 624 may be gradual, or may be an abrupt interface. In some embodiments, constriction 624 may have a length of between 20 and 50 μm, for example 40 μm. Ports 621 and 623 are formed by etching the base 606 to reach the second flow channel 620 and are configured for circulation of a first fluid containing cells. Ports 631 and 633 are also formed by etching the base 606 to reach the second flow channel 620 and are configured for circulating a second fluid for injection to a cell via nanoneedle.

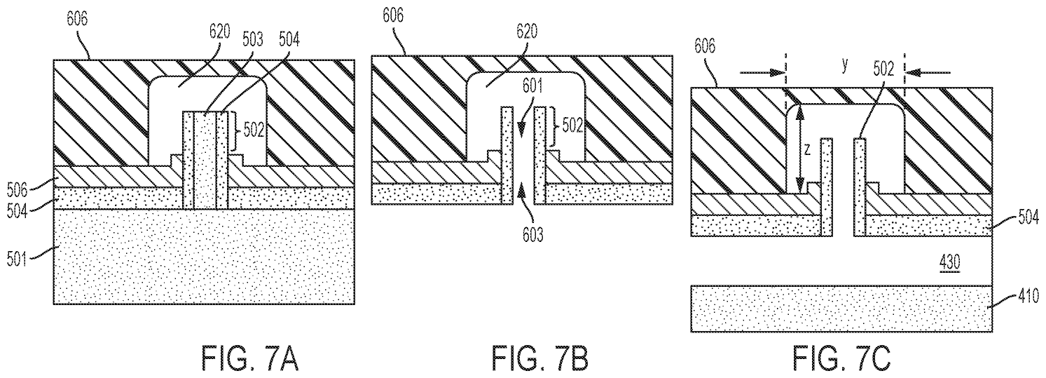

FIGS. 7A to 7C are schematic diagrams illustrating an exemplary fabrication sequence to form a nanoneedle and attach the nanoneedle to the first and second flow channels, according to some aspects.

In FIG. 7A, base 606 is bonded over a top surface of layer 506, by first bringing the completed structure shown in FIG. 5E and the completed structure shown in FIG. 6B together and carefully aligning the constriction 624 of the first flow channel 620 in base 606 with the nanoneedle 502. In some embodiments, bonding of PDMS base 606 over PDMS layer 506 may be facilitated by an oxygen plasma treatment to enhance adhesion between the two materials. It should be appreciated that poor lateral alignment may lead to breakage of nanoneedle 502 when cover 606 is bonded over layer 506. In some embodiments, the width y of first flow channel 620 may be selected to account of bonding alignment, and should increase compared to a single nanoneedle apparatus if multiple nanoneedles are provided.

In FIG. 7B, both wafer 501 and nanowire 503 are removed by a suitable etching process. In one embodiment, wafer 501 and nanowire 503 are both Ge, and are removed in a wet etching solution comprising H₂O₂. It should be appreciated that after nanowire 503 is removed, the side wall material 504 forms the structure of nanoneedle 502 and extends between a first opening 601 and a second opening 603.

In FIG. 7C, substrate 410 is bonded under a bottom surface of base 504, by bringing the completed structure shown in FIG. 7B and the completed structure as shown in FIG. 4B and carefully aligning the second flow channel 430 in substrate 410 with the nanoneedle 502.

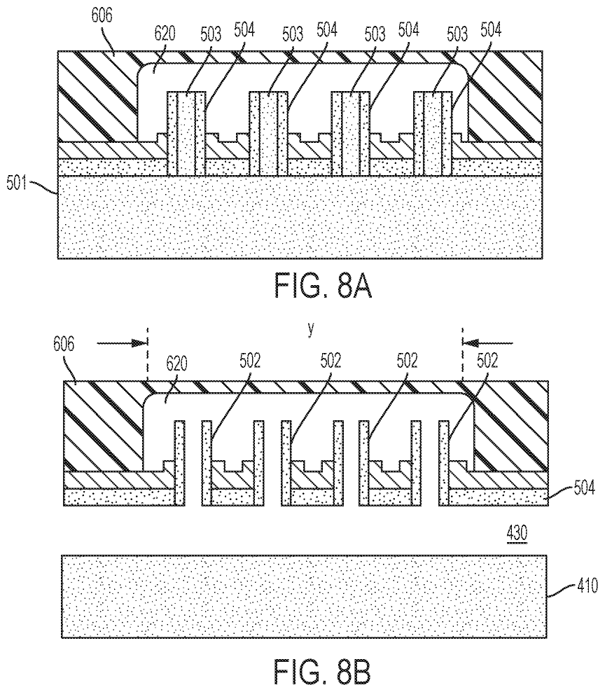

FIGS. 8A and 8B are schematic diagrams illustrating a cross-section view of a fabrication sequence to form a nanoneedle array and attach the nanoneedle array to the first and second flow channels, according to some embodiments of the present application. FIG. 8A shows a base 606 of the completed structure shown in FIG. 5E bonded over layer 506 having a nanowire array 503. Both wafer 501 and nanowire 503 are removed by a suitable etching process. In one embodiment, wafer 501 and nanowire 503 are both Ge, and are removed in a wet etching solution comprising H₂O₂. After etching nanowires 503 away, the sidewall 504 becomes nanoneedles 502.

In FIG. 8B, substrate 410 is bonded under a bottom surface of base 504, such that the array of nanoneedles 502 are disposed in the second flow channel 430. The width y of first flow channel 620 may be selected to accommodate the multiple nanoneedles in the nanoneedle array 502.

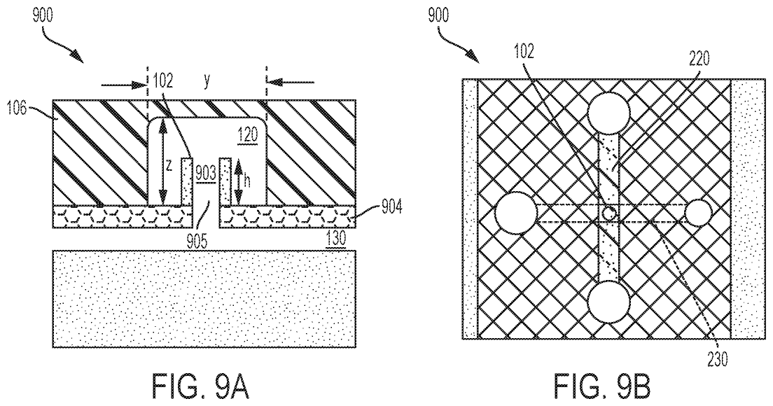

FIGS. 9A is a schematic diagrams illustrating a cross-sectional view of an apparatus 900, according to an alternative embodiment. Apparatus 900 is similar to apparatus 100 shown in FIG. 1B in many aspects, with one difference being that nanoneedle 102 is disposed on and supported by a substrate 904. Substrate 904 comprises an opening 905 that coincides with a bottom opening 903 of nanoneedle 102. FIG. 9B is a schematic diagrams illustrating a top view of apparatus 900.

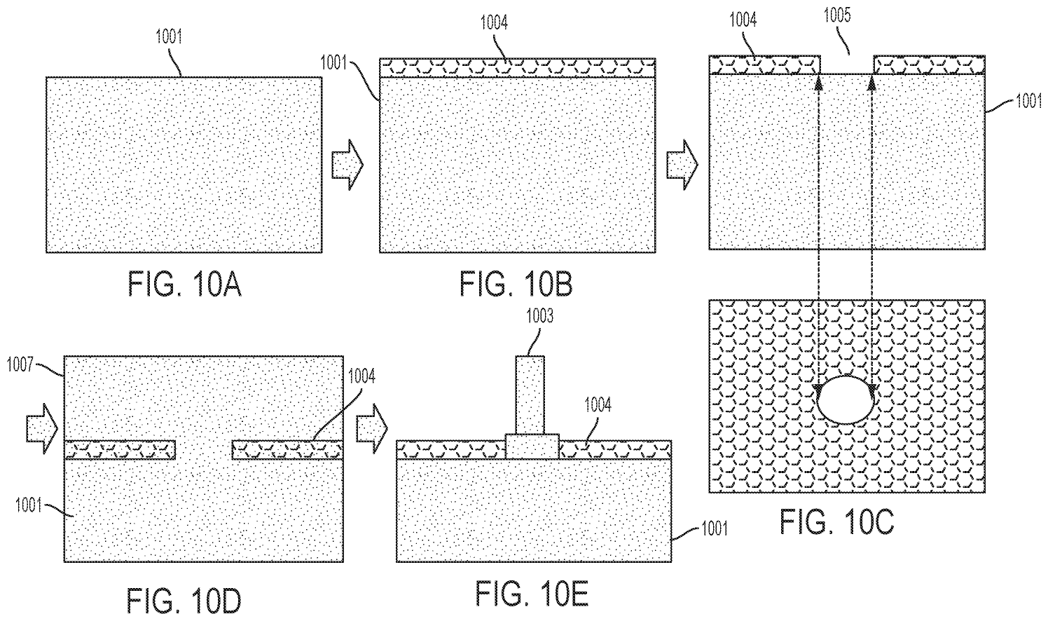

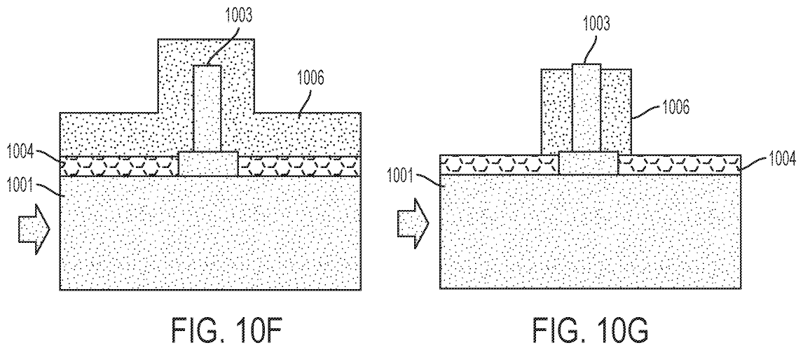

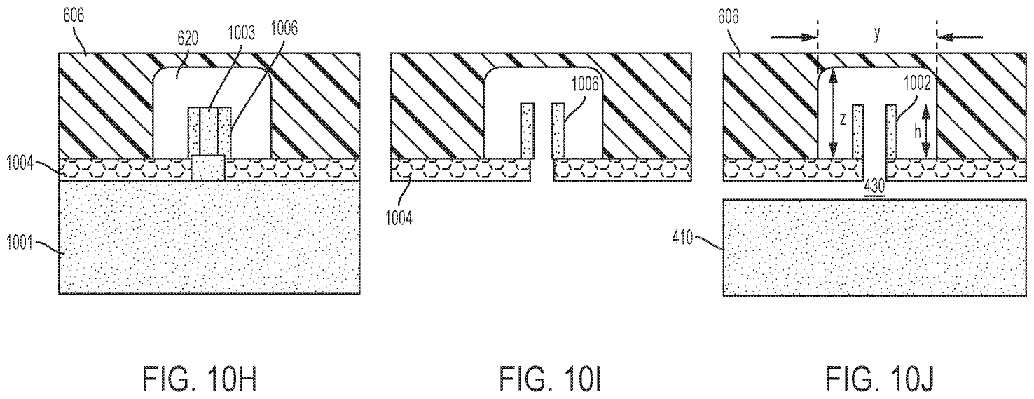

FIGS. 10A to 10J are schematic diagrams illustrating an exemplary fabrication sequence to form apparatus 900, according to some aspects. The process may start with a Ge wafer 1001 as shown in FIG. 10A. In FIG. 10B, a dielectric layer 1004 is deposited over the top surface of Ge wafer 1001. Dielectric layer may be any suitable semiconductor material. In some embodiments, dielectric layer is silicon nitride. In one example, about 500 nm thick silicon nitride is deposited via a chemical vapor deposition process as dielectric layer 1004.

In FIG. 10C, an opening 1005 is formed in dielectric layer 1004, using photolithography or electron beam lithography with a suitable etching technique such as RIE. The lateral size or diameter of the opening 1005 may be for example 150 nm.

Proceeding to FIG. 10D, additional semiconductor material 1007 are deposited to cover the dielectric layer 1004 and opening 1005. In some embodiments, semiconductor material 1007 is Ge and may be deposited using a suitable gas phase deposition process such as chemical vapor deposition (CVD) or atomic layer deposition.

In FIG. 10E, Ge nanowire 1003 are formed by etching the deposited Ge material 1007. Ge nanowire 1003 may have any suitable diameter or shape that will define the size and shape of the inner diameter of an eventual nanoneedle. In one example, Ge nanowire 1003 has a diameter of 100 nm.

In FIG. 10F, a conformal layer 1006 is deposited and covers the top of dielectric layer 1004as well as side wall and top of nanowire 1003. In some embodiments, conformal layer 1006 is CVD deposited silicon oxide. In one non-limiting example, the thickness of silicon oxide 1006 is 150 nm.

In FIG. 10G, a RIE etch is performed to selectively etch back a portion of layer 1006 over the top of nanowire 1003, such that the nanowire 1003 is exposed on its top surface.

In FIG. 10H, base 606 with the first flow channel is bonded over a top surface of dielectric layer 1006. In FIG. 10I, the Ge wafer 1001 and nanowire 1003 are removed by a suitable Ge etching process such as using a wet etching solution comprising H₂O₂. It should be appreciated that after nanowire 1003 is removed, the side wall material 1006 forms the structure of nanoneedle 1002 as shown in FIG. 10J. Also shown in FIG. 10J, substrate 410 with second flow channel 430 is bonded under a bottom surface of dielectric layer 1004.

The inventor has appreciated and acknowledged that in the alternative apparatus 900, smaller dimensions for nanoneedle and the constriction inside the first flow channel may be provided using precise nanolithography techniques such as electron beam lithography. For example, as shown in FIG. 10J, the height z and width y of the first flow channel at its narrowest point may be between 5 μm and 10 μm. The height h of the nanoneedle 1002 may be selectively controlled by the thickness of deposited Ge material 1007, and may be between 1 μm and 5 μm, or between 2 μm and 4 μm.

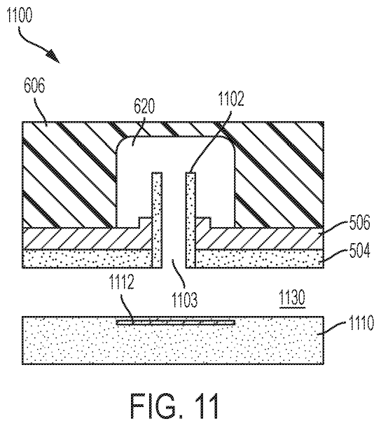

FIG. 11 is a schematic diagram showing a cross-sectional view of an apparatus 1100 with a nanopump 1102 and a piezoelectric membrane driver 1112, according to an aspect of the present application. Piezoelectric membrane driver 1112 may be formed near a surface of substrate 1110 facing the opening 1103 of the nanoneedle 1102, and configured to exert pressure to liquid in the second flow channel 1130 to control injection and extraction of fluid from a cell penetrated by nanoneedle 1102 in the first flow channel 620.

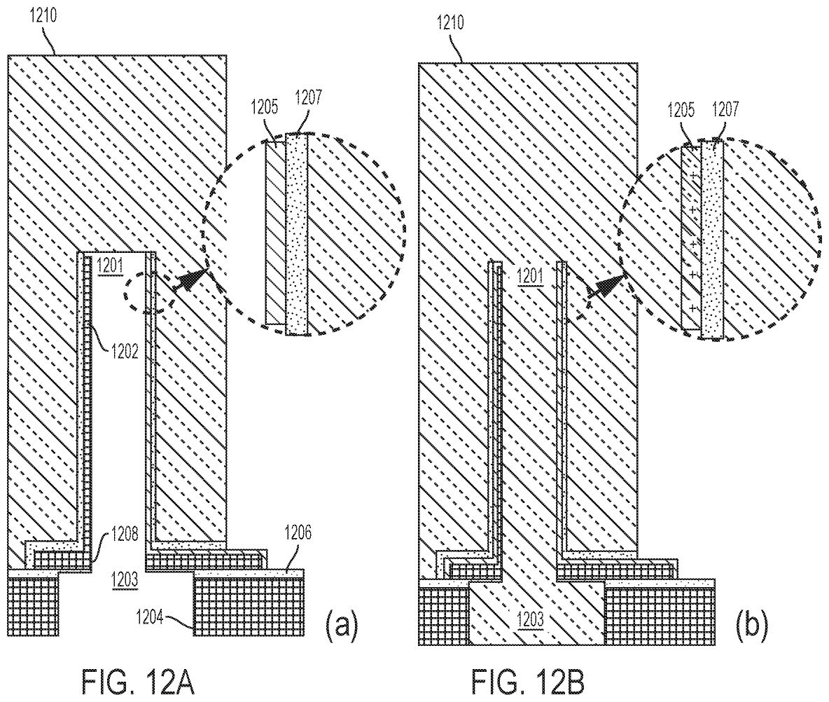

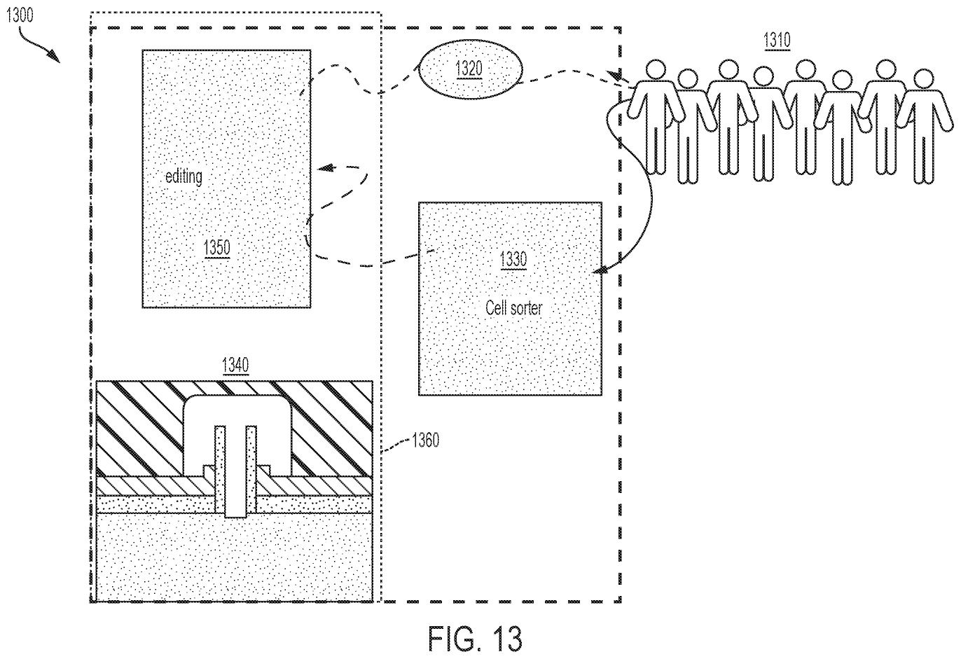

FIG. 12A is a schematic diagram showing a cross-sectional view of a nanopump 1202, according to an aspect of the present application. Nanopump 1202 consists of side wall material 1202 and extends from a semiconductor or polymer substrate 1204. A dielectric 1206 or other layer may be formed on the substrate, over which the base 1208 of nanoneedle 1202 may be formed. The sidewall 1205 of the nanoneedle 1202 may be formed from any of the aforementioned materials from which a nanoneedle may be fabricated. A fluoropolymer (e.g., Teflon) or other hydrophobic (e.g. hydrophobic or superhydrophobic polymers) coating 1207 may be applied onto the outer surface of the nanoneedle sidewall 1205. The coating may comprise one or more thin films that can be formed, for example, by vapor deposition or solution chemical reactions. In one particular embodiment the nanoneedle sidewall 1205 may be formed from a conductive material such as metal, for example from gold, silver, copper or titanium. A counter electrode (not shown) may be provided in contact with liquid in a flow channel where nanoneedle 1202 is placed, such that a voltage may be applied between the conductive side wall 1205 of nanoneedle 1202 and the counter electrode. The voltage can be used to control the intake and ejection of fluid 1210 from the opening 1201 of nanoneedle 1202.

Liquid 1210 may be an intracellular fluid, when the opening 1201 of nanoneedle 1202 is placed inside a cell or a cell nucleus after penetration by the nanoneedle 1202. FIG. 12A shows the nanopump 1202 when no bias voltage is applied. In FIG. 12A, little to no liquid 1210 will enter inside nanoneedle 1202 via opening 1201, because the outer surface of nanoneedle 1202 is generally hydrophilic.

FIG. 12B is a schematic diagram showing a cross-sectional view of nanopump 1202 under a bias voltage, according to an aspect of the present application. When a bias voltage is applied between the conductive side wall 1205 and the counter electrode, a positive charge is established on the conductive side wall 1205 that forms the nanoneedle 1202 and fluid 1210 is drawn into the the nanoneedle 1202 via opening 1201 through the bottom opening 1203 by electrowetting effect as well as capillary action. Nanoneedle 1202 may then be withdrawn from inside of the cell with the bias voltage being continually applied. After withdrawal, the bias voltage may then be removed, causing the fluid to be expelled from the nanoneedle 1202 as a result of capillary action and the hydrophobic nature of its outer surface.

FIG. 13 is a schematic diagram showing an exemplary medical system 1300 that includes a nanopump apparatus 1340, according to an aspect of the present application. Subject 1310 may be a patient, a user, an operator of medical system 1300, or in some embodiment both a patient and an operator of medical system 1300 for self-diagnostic or therapeutic uses. Apparatus 1340 may be a nanopump having a nanoneedle, similar to the embodiments discussed above in relation to FIGS. 1A through FIG. 12B. Apparatus 1340 may be disposed within a device 1360, along with an editing unit 1350. A sample such as blood or bodily fluid sample from subject 1310 may be processed in cell sorter 1330, where cells are separated with high throughput and high selectivity, although other types of cell processing apparatus may also be used. A fluid containing cells of interest is delivered or circulated from cell sorter 1330 to device 1360. Within device 1360, editing unit 1350 may perform functions such as adding chemical drug compounds, editing DNA, RNA, lipid, protein, or genomes using a variety of means known in the art, such as CRISPR. Editing unit may process cells of interest using nanopump apparatus 1340 with a method as discussed in any of the preceding paragraphs. Device 1360 may, as a result of processing in editing unit 1350, output one or more edited cells to cell culture unit 1320. An output of cell culture unit 1320 may be infused back to subject 1310 as a therapeutic solution to one or more diseases in subject 1310.

In some embodiments, one or more components within the device 1360 may be a consumable component that allows flexibility in reconfiguring the device 1360 for new applications with the same or different set of configurations. For example, the nanopump apparatus 1340 may be provided in the form of a consumable cartridge, such that a different nanopump apparatus with different nanoneedle configurations such as needle dimensions, or biochemical functionalizations may be used to replace a previously used nanopump apparatus cartridge, without the need to change the entire device 1360. Such a consumable cartridge may reduce the cost for configuring and reconfiguring the medical system 1300.

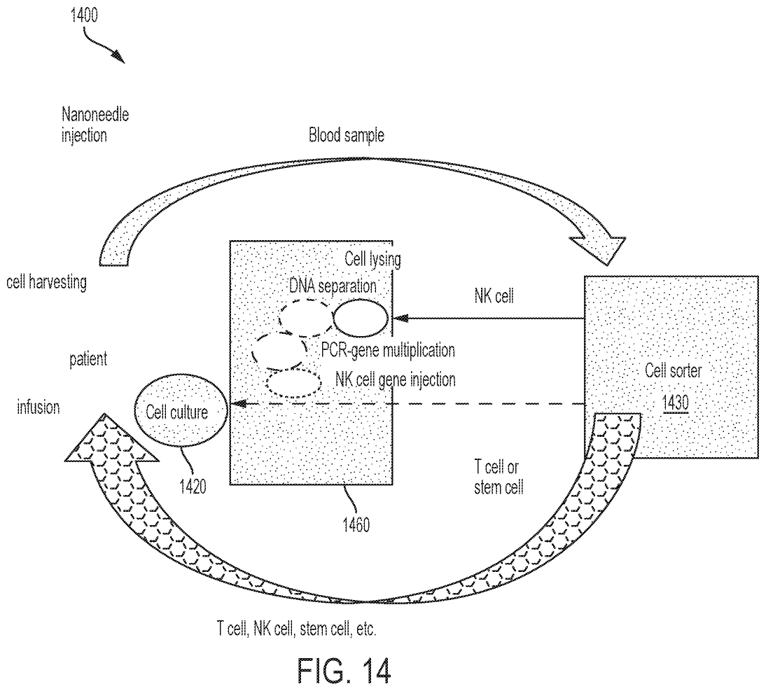

FIG. 14 is a schematic diagram showing an exemplary medical system 1400, according to another aspect of the present application. In the medical system 1400, a cell sorter 1430 may screen and separate a particular groups of cells of interest, such as T-cell, NK cells, stem cell, etc. A device 1460 may use a nanopump apparatus such as those described in any of the preceding paragraphs to perform a method of cell lysing, DNA separation, PCR-gene multiplication or NK cell gene injection. After the output from device 1460 is processed in cell culture unit 1420, the output of cell culture unit 1420 may be infused back to a patient as a therapeutic solution to one or more diseases.

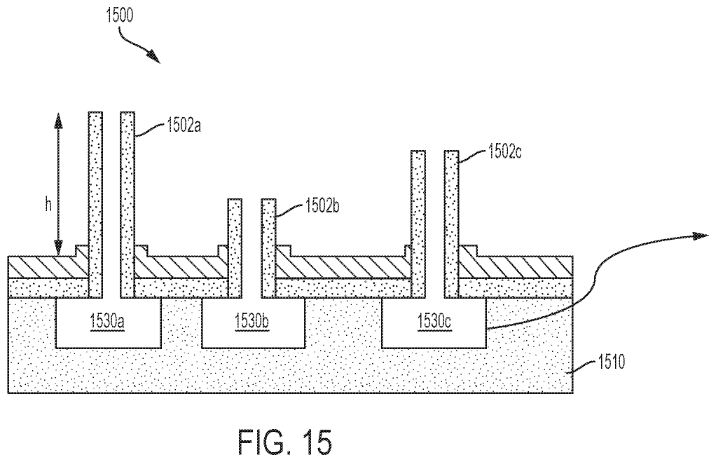

FIG. 15 is a schematic diagram showing an exemplary apparatus 1500, according to an aspect of the present application. Apparatus 1500 comprises multiple nanoneedles 1502a, 1502b, 1502c each connected to respective second flow channels 1530a, 1530b and 1530c that are distinct and separated from each other, in order to enable individualized injection and/or extraction based on fluids in the separate second flow channels connected at the bottom openings of the nanoneedles. In the embodiment shown in FIG. 15, multiple nanoneedles 1502a, 1502b, 1502c have different heights, and are configured to penetrate into different depths when a cell in a first flow channel is attached to the multiple nanoneedles 1502a, 1502b, 1502c, or to penetrate into different depths of more than one cells. The substrate 1510 may be formed of a flexible and stretchable material such as polymer or fabric, such that the apparatus 1500 may be a flexible array for brain research, for example by electrically or electrochemically stimulating cells/tissues of a brain. Furthermore, such an apparatus may also enable injection of drug, gene, neurotransmitters, etc. into the cells or tissues of the brain.

The inventor has appreciated and acknowledged that fabrication of functional devices such as those described in the present application may allow brain research and neurological disease treatments. While each of the nanoneedles 1502a-1502c may be a nanopump, one or more of flow channels 1530a-1530c may be connected to an external micropump to perform fluidic pumping.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Further, though advantages of the present invention are indicated, it should be appreciated that not every embodiment of the technology described herein will include every described advantage. Some embodiments may not implement any features described as advantageous herein and in some instances one or more of the described features may be implemented to achieve further embodiments. Accordingly, the foregoing description and drawings are by way of example only.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the invention may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Such alterations, modifications, and improvements are intended to be part of this disclosure, and are intended to be within the spirit and scope of the invention. Further, though advantages of the present invention are indicated, it should be appreciated that not every embodiment of the invention will include every described advantage. Some embodiments may not implement any features described as advantageous herein and in some instances. Accordingly, the foregoing description and drawings are by way of example only.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

I claim:

1. An apparatus comprising:
a first flow channel configured to accommodate a first fluid containing a cell circulating along a first flow direction;
a nanoneedle disposed in the first flow channel and configured to penetrate the cell, wherein,
the first flow channel comprises a constriction adjacent the nanoneedle.

2. The apparatus of claim 1, wherein:
the first flow channel has a first portion within the constriction and a second portion outside the constriction, the first portion has a first size along a first direction perpendicular to the first flow direction and the second portion has a second size along the first direction,
wherein the first size is smaller than the second size.

3. The apparatus of claim 2, wherein:
the first size is configured to direct the cell in the first flow channel to be penetrated by the nanoneedle.

4. The apparatus of claim 2, wherein:
the first size is between 0.25 and 5 times the average diameter of the cell.

5. The apparatus of claim 2, wherein:
the first size is between 0.5 and 2 times the average diameter of the cell.

6. The apparatus of claim 2, wherein:
the first size is between 0.25 and 200 μm.

7. The apparatus of claim 2, wherein:
the first size is between 0.5 and 100 μm.

8. The apparatus of claim 1, wherein:
the cell is an animal cell, a plant cell, a bacteria cell or a fungi cell.

9. The apparatus of claim 1, wherein:
the cell comprises a nucleus and wherein the nanoneedle is configured to penetrate the nucleus of the cell.

10. The apparatus of claim 1, wherein:
the cell comprises a nucleus and wherein the nanoneedle is configured to penetrate a membrane of the cell.

11. The apparatus of claim 1, further comprising a second flow channel configured to accommodate a second fluid, and wherein
the nanoneedle comprises a first opening disposed in the first flow channel and a second opening disposed in the second flow channel, wherein the second flow channel is configured to be in fluidic communication with the first flow channel via the nanoneedle.

12. The apparatus of claim 1, further comprising a second flow channel configured to accommodate a second fluid, and wherein
the nanoneedle comprises a first opening disposed in the first flow channel and a second opening disposed in the second flow channel, wherein the second flow channel is configured to be in fluidic communication with an interior of the cell via the nanoneedle.

13. The apparatus of claim 12, further comprising a mechanical actuator disposed in the second flow channel and configured to inject fluid into or extract fluid from the interior of the cell via the nanoneedle.

14. The apparatus of claim 12, wherein the nanoneedle comprises a nanopump with a conductive sidewall, the nanopump configured to inject fluid into or extract fluid from the interior of the cell via the nanoneedle.

15. The apparatus of claim 1, wherein:
the nanoneedle comprises a first nanoneedle segment configured to be exposed to the first fluid in the first flow channel and having a length of between 0.5 and 100 μm.

16. The apparatus of claim 15, wherein:
The first nanoneedle segment comprises a first opening having an average outer size of between 5 and 1000 nm.

17. The apparatus of claim 15, wherein:
The first nanoneedle segment comprises a first opening having an average inner size of between 5 and 1000 nm.

18. The apparatus of claim 1, wherein:
the first portion of the first flow channel is transparent to an optical microscope illumination wavelength.

19. The apparatus of claim 1, wherein the nanoneedle is a first nanoneedle and the apparatus further comprises:
a second nanoneedle disposed in the first flow channel and configured to penetrate the cell.

20. The apparatus of claim 12, wherein the nanoneedle is a first nanoneedle and the apparatus further comprises a third flow channel configured to accommodate a third fluid, and
a second nanoneedle disposed in the first flow channel and configured to penetrate the cell, wherein the second nanoneedle comprises a third opening disposed in the first flow channel and a fourth opening disposed in the third flow channel, wherein the third flow channel is configured to be in fluidic communication with an interior of the cell via the second nanoneedle.

21. A method of manufacturing a nanopump apparatus, the method comprising:
forming a nanoscale wire;
forming a side wall material surrounding the nanoscale wire;
disposing the nanoscale wire inside a first flow channel;
subsequent to disposing the nanoscale wire inside the first flow channel, selectively removing the nanowire to form a nanoneedle from the side wall material.

22. The method of claim 21, wherein:
forming the nanoscale wire comprises forming the nanoscale wire on a semiconductor substrate, such that the nanoscale wire is elongated along a direction substantially perpendicular to a planar surface of the semiconductor substrate.

23. The method of claim 22, wherein the nanoscale wire has a first end supported by the semiconductor substrate and a second end distal to the first end, the side wall material comprises a first portion surrounding the first end of the nanoscale wire and a second portion surrounding the second end of the nanoscale wire, the method further comprising:
embedding the first end of the nanoscale wire and the first portion of the side wall material in a support layer with a flow channel attach surface facing away from the planar surface of the semiconductor substrate.

24. The method of claim 23, wherein:
disposing the nanoscale wire inside the first flow channel comprises coupling the flow channel attach surface of the support layer to the first flow channel such that the first end of the nanoscale wire is disposed inside the first flow channel.

25. The method of claim 24, the method further comprising:
removing the semiconductor substrate.

26. The method of claim 21, the method further comprising:
subsequent to removing the nanowire to form a nanoneedle, exposing the nanoneedle to a second flow channel, such that the second flow channel is in fluidic communication with the first flow channel via the nanoneedle.

27. The method of claim 21, wherein selectively removing the nanoscale wire to form a nanoneedle from the side wall material comprises a selective wet etch of the nanoscale wire.

28. The method of claim 21, wherein the nanoneedle is a first nanoneedle and the method comprises:
forming a second nanoneedle;
disposing a first end of the first nanoneedle and a first end of the second nanoneedle in the first flow channel.

29. The method of claim 28, further comprising:
disposing a second end of the first nanoneedle in a second flow channel, such that the second flow channel is in fluidic communication with the first flow channel via the first nanoneedle;
disposing a second end of the second nanoneedle in a third flow channel, such that the third flow channel is in fluidic communication with the first flow channel via the second nanoneedle.

30. A method of operating an apparatus including a first flow channel, a second flow channel, a nanoneedle that comprises a first opening disposed in the first flow channel and a second opening disposed in the second flow channel, wherein the first flow channel comprises a constriction adjacent the nanoneedle, the method comprising:
circulating a first fluid containing a cell along a first flow direction in the first flow channel;
penetrating the cell with the nanoneedle;
accommodating a second fluid containing a reagent in the second flow channel; and
delivering the reagent from the second fluid into the cell via the nanoneedle.

31. The method of claim 30, wherein:
the first flow channel has a first portion within the constriction and a second portion outside the constriction, the first portion has a first size along a first direction perpendicular to the first flow direction and the second portion has a second size along the first direction,
wherein the first size is smaller than the second size.

32. The method of claim 30, further comprising:
delivering the reagent from the second flow channel into a nucleus of the cell.

33. The method of claim 30, further comprising:
delivering the reagent from the second flow channel through a membrane of the cell.

34. The method of claim 30, further comprising:
circulating the cell within the first flow channel with a predetermined circulation timing;
controlling the delivering of the reagent with a timing and dosage based at least in part on the predetermined circulation timing.

35. The method of claim 30, the method comprising:
delivering an analyte from the cell to the second flow channel.

36. The method of claim 30, wherein:
the cell is viable subsequent to penetrating the cell with the nanoneedle.

37. The method of claim 30, further comprising:
subsequent to penetrating the cell with the nanoneedle, removing the nanoneedle from the cell such that the cell is recirculated in the first fluid.

38. The method of claim 30, wherein the nanoneedle is a first nanoneedle and the apparatus further comprises a second nanoneedle disposed in the first flow channel, the method further comprising:
subsequent to penetrating the cell with the nanoneedle, penetrating the cell with the second nanoneedle.

39. A medical system, comprising:
a nanopump comprising a first flow channel, a second flow channel having a fluid containing a reagent, a nanoneedle that comprises a first opening disposed in the first flow channel and a second opening disposed in the second flow channel, wherein:
the first flow channel is configured to receive a first sample containing a cell from a user,
the nanoneedle is configured to penetrate and deliver the reagent inside the cell.

40. The medical system of claim 39, wherein the nanopump is configured to deliver a second sample to the user.

41. The medical system of claim 39, further comprising:
a cell sorter configured to receive a bodily fluid from the user and to deliver the first sample containing the cell to the first flow channel.

42. The medical system of claim 41, wherein:
the nanopump and the cell sorter are disposed in a housing.
